(19) United States
(12) Patent Application Publication
(10) Pub. No.: US 2013/0319059 A1
(43) Pub. Date: Dec. 5, 2013

(54) INTEGRATED CARBON CAPTURE AND ALGAE CULTURE

(71) Applicants: Shulin Chen, Pullman, WA (US); Zhanyou Chi, Pullman, WA (US); Yuxiao Xie, Pullman, WA (US); Baisuo Zhao, Pullman, WA (US)

(72) Inventors: Shulin Chen, Pullman, WA (US); Zhanyou Chi, Pullman, WA (US); Yuxiao Xie, Pullman, WA (US); Baisuo Zhao, Pullman, WA (US)

(73) Assignee: WASHINGTON STATE UNIVERSITY, Pullman, WA (US)

(21) Appl. No.: 13/992,291

(22) PCT Filed: Dec. 9, 2011
(86) PCT No.: PCT/US11/64127

§ 371 Date: Aug. 19, 2013

Related U.S. Application Data
(60) Provisional application No. 61/421,449, filed on Dec. 9, 2010.

(51) Int. Cl. C12M 1/00; C05F 11/00; C12P 19/04; C12P 7/64; C12P 23/00

(52) U.S. Cl. CPC C12M 21/02; C12P 7/64; C12P 23/00; C12P 19/04; C05F 11/00
USPC 71/23; 435/134; 435/67; 435/101; 435/289.1; 435/303.1

(57) ABSTRACT

The feasibility of using CO₂ from a concentrated source to grow microalgae is limited by the high cost of CO₂ capture and transportation, as well as significant CO₂ loss during algae culture. Another challenge is the inability of algae in using CO₂ during night while CO₂ is continuously produced from the source. To address these challenges, this invention provides a process in which CO₂ is captured as bicarbonate and used as feedstock for algae culture. Then the carbonate is regenerated in the algae culture process as absorbent to capture more CO₂, which is converted to bicarbonate for use as feedstock, etc. This process significantly reduces carbon capture costs since it avoids the energy for carbonate regeneration. Also, transporting a solid or aqueous bicarbonate solution has a much lower cost than transporting compressed CO₂, and using bicarbonate provides a better alternative for CO₂ delivery to algae culture systems than supplying CO₂ gas.

### BACKGROUND OF THE INVENTION

1. Field of the Invention

The invention generally relates to integrated methods and systems for utilizing CO₂ as a feedstock for microorganisms. In particular, the invention provides methods for capturing CO₂, converting it to bicarbonate, and using the bicarbonate as a carbon source for the growth of photosynthetic algae and cyanobacteria.

2. Background of the Invention

### Challenges to Capture CO₂ for Algae Culture

Combustion of fossil fuels such as coal, petroleum, and natural gas for energy is the major reason for the increased CO₂ concentration in the atmosphere, and this has caused growing concern with respect to the effects on global climate change and ocean acidification (Iglesias-Rodriguez et al., 2008). Usually, the production of 1 kWh of electricity leads to 0.95 kg CO₂ emission from coal combustion (DOE&EPA, 2000). A small 50 MW coal fired power plant produces about 1,140 metric ton (MT) CO₂/day, whereas a mid-sized 500 MW plant produces 11,400 MT CO₂/day (EPA, 2011).

One potential way to reduce this emission is to capture, transport, and store CO₂ in geologic formations. However, compared to processes without carbon capture, the coal combustion process with carbon capture and storage has a very high cost, and can become a favored technology only if the emission price of CO₂ reaches $67/MT (NETL, 2010; Plasynski et al., 2009). Also, storage of CO₂ in geologic formations may create new environmental issues such as induction of earthquake activity, threat of CO₂ leakage, or potential contamination of groundwater (Plasynski et al., 2009; Sminchak and Gupta, 2001).

Instead of storage in geologic formations, an ideal solution for captured CO₂ would be conversion into biomass, so that CO₂ can be recycled into the biotic carbon pool, or stored in soil carbon pools as organic or inorganic carbon (Lal, 2004; Lee et al., 2010; Ramanan et al., 2010). Production of biofuel from the grown biomass would reduce the usage of fossil fuels, and this would likewise contribute to a reduction in CO₂ emissions (Packer, 2009; Pienkos and Darzins, 2009).

Biodiesel can be produced from a variety of traditional oil crops such as soybeans, canola, palm, corn, and jatropha. However, these crops compete with food resources, and may suffer from production limitations in the future. Microalgae culture promises a superior alternative, due to its significant advantages for high productivity, no competition with food sources, as well as generating valuable co-products (Chen et al., 2010; Chisti, 2007). However, key challenges such as the high cost of algal biomass production, harvesting, and oil extraction remain to be solved before such cultures are ready for industrial application.

The high cost of feedstock CO₂ is the major obstacle for algal biomass production. All current carbon capture technologies require large amounts of extra energy to regenerate the absorbent, and this leads to a significantly decreased power plant efficiency and corresponding increased cost of electricity (COE). For example, based on the reaction of CO₃²⁻+CO₂+H₂O [symbol] 2HCO₃⁻, the Benfield™ process of Honeywell Group's UOP Inc. developed a process that uses a high concentration of potassium carbonate to absorb CO₂, and convert it into potassium bicarbonate (Plasynski et al., 2009). The bicarbonate is then converted back to carbonate by releasing CO₂ using heat. This process consumes 1,381-2,549 MJ of extra thermal energy to remove 1 MT CO₂ (Furukawa and Bartoo, 1997), and this parasitic energy consumption is about 36.4%-67.3% of the electricity produced.

Usually, the available land around power plants is limited, and thus CO₂ has to be captured and transported to algae ponds a long distance away. However, this is limited by high costs for carbon transportation. Typically, CO₂ is compressed to a pressure of 150 atm to be transported through a pipeline. This compression process consumes considerable energy and increases the transportation cost. The cost estimated by Kadam et al (1997) for 100 km-transportation is $8.48/MT CO₂ for compression and drying, as well as $3.30/MT CO₂ for pipeline transportation, respectively.

Using captured carbon for algae culture also faces other major challenges. For example, the captured CO₂ cannot be temporarily stored during night time or winter, when algae do not grow. Also, there is a significant loss of CO₂ from outgas if the algae are cultured in an open system. As a result of these problems a maximum of only 25% CO₂ is typically captured by algae culture (Benemann, 2009). This is not satisfactory for a successful carbon capture process, which requires that 90% of the CO₂ in flue gas be recovered (Benemann, 2009; NETL, 2010).

In summary, current technology for using CO₂ from a concentrated source for algae culture is limited by the high cost of carbon capture, high cost of transportation, difficulty of CO₂ temporary storage, and low efficiency. An alternative process for CO₂ capture, transport, and delivery is required for an industrial scale algal biomass production system.

### SUMMARY OF THE INVENTION

The invention provides integrated methods and systems for capturing CO₂ and converting captured CO₂ to bicarbonate, transporting bicarbonate e.g. into an alkaliphilic algae or cyanobacteria culture system, where the bicarbonate serves as a carbon source for the microorganisms, and recycling medium from the culture system, which contains a high concentration of dissolved CO₂) by recapturing the CO₂ and converting it to bicarbonate, which is then used in the alkaliphilic algae or cyanobacteria culture system, and so on. The CO₂ is thus recycled indefinitely. If the original source of the CO₂ is the culture system, then the method is truly a closed loop method. However, the initial input of CO₂ (or subsequent inputs) may be from other sources (e.g. industrial sources), in which case the method is partially closed, but may continue as a closed loop system. Also, the bicarbonate (which may be in either solid or solution form) may serve as the sole source of carbon for the microorganisms, or, alternatively, other carbon sources may also be used.

Systems for carrying out the method are also provided. The systems include apparatuses or means for i) capturing and ii) converting the CO₂, a suitable culture system, and integrated means for transporting the CO₂ and bicarbonate from one system component to another. The methods and systems are advantageous in part because the transport of bicarbonate (either as a solid or in solution) is less costly and less dangerous than the transport of CO₂ gas.

### BRIEF DESCRIPTION OF THE DRAWINGS

FIG. 1: System overview for the integrated carbon capture and algae culture process.

FIGS. 2A and B. Dunaliella primolecta strains growth with sodium bicarbonate as solely carbon source (a) pH variation in the culture (b) optical distribution.

FIGS. 3A and B. Dunaliella primolecta strains growth with different inorganic carbon supply methods. (a) pH variation in the culture (b) optical distribution.

FIGS. 4A and B. Euhalothece ZM001 growth with 1 M sodium bicarbonate solution (a) pH variation in the culture (b) optical distribution.

### DETAILED DESCRIPTION

### Handling Captured Carbon as Aqueous Solution

Upon analysis of these CO₂ transportation related problems, it can be deduced that these problems exist because captured carbon is handled as a compressed CO₂, rather than water solution at normal atmospheric pressure. Fortunately, inorganic carbon (Ci) not only exists as CO₂ gas, but also as carbonate or bicarbonate salts. The solubility of certain carbonate salts in water is very high. For example, the solubility of sodium bicarbonate at 25° C. is 103 g/L or 10.3% (w/v). If the captured carbon is converted into a bicarbonate/carbonate aqueous solution, it can easily be transported in a water pipeline at normal pressure.

Zhou and Richard (2005) estimated the cost for a 100 km horizontal transport of water to be $0.05-$0.06/m³ by canal and $ 0.104-$0.125/m³ by water tunnel (2005). It can be predicted that a bicarbonate solution will have a much lower transportation cost than the corresponding compressed CO₂. Also, the transportation cost of a water solution can be linearly reduced if the transport distance is shortened, whereas compression is obligatory for any distance of CO₂ gas transportation.

For an algae culture process, the bicarbonate water solution can be stored during winter or night time, and supplied to the algae culture system in summer or day time. For example, the daily emission of 1,140 tons of CO₂ from a small 50 MW power plant can be stored as a 22,800 m³ sodium bicarbonate solution. It may be noted that delivery of this bicarbonate solution to an algae culture system does not require a gas sparging system. Also, algae culture at high pH would prevent invading and undesirable species from contaminating the designated culture systems.

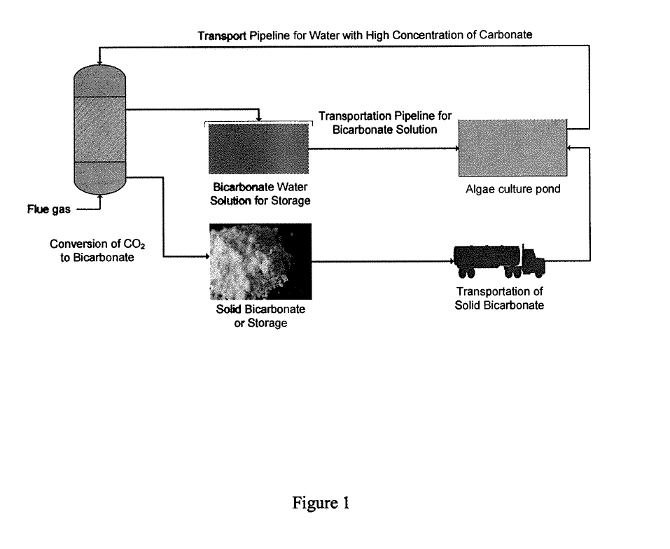

There are actually many methods of CO₂ capture that convert CO₂ into bicarbonate, and all these methods can be used as a method in this integrated system. If the bicarbonate is produced as solids, it can be stored and/or transported as solids, which will save significant cost for compression of CO₂. If bicarbonate is produced as water solution, it can be stored and/or transported with water pipeline, or open water channel, as indicated in FIG. 1. This also saves the cost for compression of CO₂. For storage and transportation purpose, higher concentration of bicarbonate in the water solution is preferred, since this will reduce the volume of bicarbonate solution to be transported.

### Bicarbonate as Feedstock for Photosynthesis

Either CO₂ or HCO₃⁻, once imported into the cell, accumulates mainly as HCO₃⁻. Lipid membranes are about 1000-fold more permeable to CO₂ than HCO₃⁻ and severe leakage occurs if a rapid equilibration between CO₂ and HCO₃⁻ occurs in the cytosol. Thus, HCO₃⁻ is normally held at steady-state, where its concentration can reach 20-40 mM, despite the extracellular CO₂ concentration of 15 μM in fresh water and 2 mM in seawater (Price et al., 2008).

According to the equilibrium H⁺+HCO₃⁻ [symbol] CO₂+H₂O, H⁺ is consumed in the conversion of HCO₃⁻ to CO₂, and the CO₂ is ultimately fixed by Rubisco in photosynthesis. Thus, steady-state usage of HCO₃⁻ as the original carbon source for photosynthesis leaves OH⁻ in the cell, and this has to be neutralized by H⁻ uptake from the extracellular environment. The reduction of H⁺ in the culture medium unavoidably leads to an increased pH, which subsequently changes the equilibrium between different Ci species. The pKa of HCO₃⁻ in fresh water at 25° C. and 1 atm is 10.33; thus the acid/base pair bicarbonate/carbonate can act as a strong buffer around this pH. The increased pH will ultimately result in higher ratio of CO₃²⁻ to HCO₃⁻. From this viewpoint, the algae culture process actually regenerates carbonate by means of solar energy.

### Alkaliphilic Algae and Cyanobacteria in Natural Soda Lakes

Although it appears promising, the potential for such a culture system depends on availability of strains of algae that can grow in a high concentration bicarbonate environment. To grow in this environment, the eukaryotic algae or cyanobacteria must overcome the high pH and high ion strength. Fortunately, the same challenges exist naturally in many soda lakes. Zavarzin et al have summarized the parameters of some soda lakes, and showed that their pH ranges from 8.4 to 10.8, and the CO₃²⁻ concentration from 0.3 to 90.2 g/L (1.5 M) (Fleming and Prufert-Bebout, 2010; Gerasimenko and Mikhodyuk, 2009; Oberholster et al., 2009; Zavarzin et al., 1999).

Even in this extreme environment, blooms of cyanobacteria can occur, and their biomass productivity can reach 10 g C/m²/day (Zavarzin et al., 1999). If the carbon content in the produced algal biomass is 50%, the dry biomass productivity would be about 20 g/m²/day, which is at the same level as an artificial open pond algae culture system designed for biofuel production (Sheehan et al., 1998). Our unpublished research on alkaliphilic cyanobacteria culture within the pH range 9.5 to 10.5 resulted in a biomass productivity of 0.1 g/L/day, which is very similar to the growth rate of other common microalgae reported to be 0.117 g/L/day (Chisti, 2007). Further efforts on culture condition optimization promise to improve this productivity.

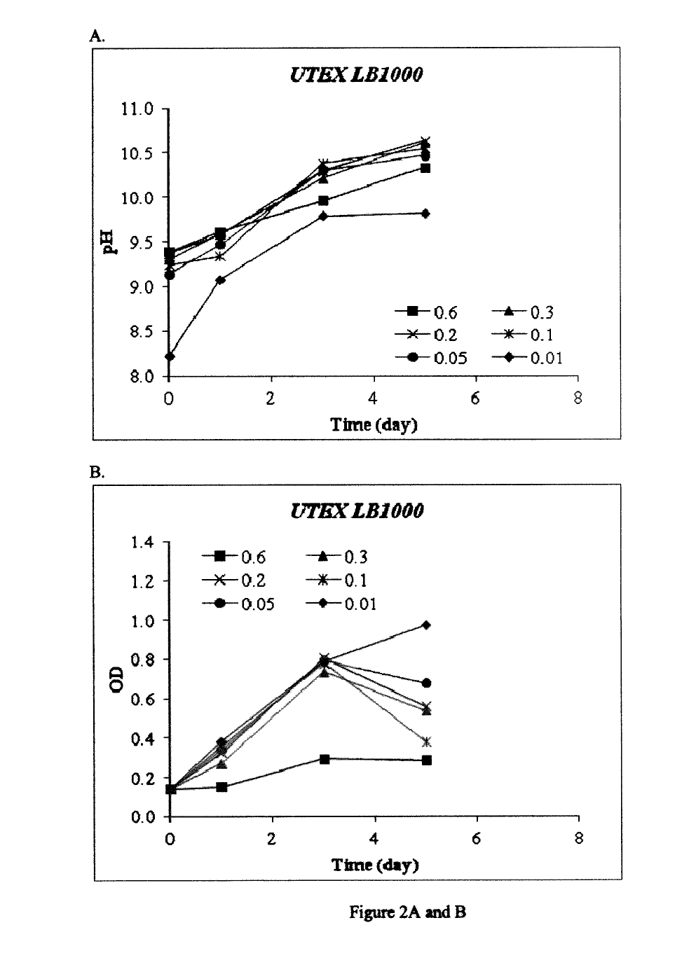

These halophilic and alkaliphilic cyanobacteria strains can be isolated and used in the integrated culture system proposed in FIG. 2. It has been reported that benthic cyanobacteria isolated from Lake Magadi include Synechocystis salina, Aphanothece stagnina, Chamaesiphon subglobosus, Rhabdoderma lineare, Synechococcus elongates, Phormidium ambiguum, Phormidium foveolarum, Phormidium retzii, Oscillatoria splendid, Sscillatoria limnetica, Spirulina fusiformis, and Spirulina laxissima. All of these strains are extreme akaliphiles, growing optimally at pH 9.9-10.4. Among them, P. orientale isolated from Lake Tuva grew optimally at pH 10.3 and 100 g/L sodium carbonate, with a total mineral salts concentration of 145 g/L (Zavarzin et al., 1999). P. ambiguum grew optimally at pH 9.9, 105 g/L sodium carbonate, with a total mineral salts concentration of 165 g/L. Additionally, Microcoleus sp. was found to be the predominate species in the cyanobacteria mat growing at pH 9.5 in Lake Khilganta. Besides these examples, eukaryotic green algae growing at pH 10.2 and a sodium carbonate concentration of 200-260 g/L have also been isolated from Lake Magadi (Zavarzin et al., 1999).

### Advantages of Closed Loop Recirculation of Carbonate for Algae Culture and Carbon Capture

Water solution with high concentration of bicarbonate is fatal for most of microbes, but some photosynthetic cyanobacteria and microalgae are able to grow in it (Mikhodyuk et al., 2008). Culture the algae or cyanobacteria that utilize bicarbonate as the carbon source for their photosynthesis and are tolerant to high concentration of bicarbonate is the key for this algae culture process.

There are some alkaliphilic algae or cyanobacteria existed in nature (Pikuta et al., 2007), which can be used in this system. More preferably are the alkaliphilic algae strains that are tolerant to high salt concentration (for example, high sodium concentration). Still more preferably are the alkaliphilic algae strains that are tolerant to high concentration of bicarbonate (for example, sodium bicarbonate). Most preferably are the algae strains that can grow in saturated sodium bicarbonate or sodium carbonate solution.

The algae or cyanobacteria strains isolated from soda lakes are ideal for this process, since soda lakes usually have high pH, high salt concentration, and high bicarbonate or carbonate concentration. Extremely alkaliphilic and halophilic algae can be isolated from this environment. These strains can be from, but not limited to, cyanobacteria such as Synechocystis sp., Cyanothece sp., Microcoleus sp., Euhalothece sp., Spirulina sp., as well as eukaryotic microalgae Chlorella and Dunaliella. Also, other algae strains with similar characters but isolated from other environment also can be used in this culture system.

The algae culture system that can be used in this system include, but not limited to, open pond system, closed photo-bioreactor system, and any other known or new designed algae culture system.

The pH in this algae culture system is from neutral (pH=7.0) to very alkaline (pH>11.0), as long as the culture algae or cyanobacteria can survive and grow. With consumption of bicarbonate in the algae culture process, the pH increases gradually.

Besides carbon capture, a major purpose of this algae culture system is to produce algal products. Microalgae are found to be a good producer for many chemicals, and it has been used as source of food and a variety of other bioproducts. These products includes, but not limited to, algae oil for biofuel, algae oil for nutraceuticals (such as omega-3 fatty acids), pigments (such as carotenoids), alginate, fertilizer, and any other bioproducts that can be produced from algae. Products produced by or from the microorganisms cultured as described herein are encompassed by the invention. Further, the methods of the invention may further comprise a step of obtaining such products from the cultured organisms, e.g. by harvesting and extricating a product, or by using harvested organisms directly in a product (e.g. fertilizer), or by extracting a product from the medium in which the organisms are grown, etc.

It is notable that high concentration of bicarbonate can produce a high density of algae biomass. It was calculated and listed in table 2. As indicated, if only 0.1 mol/L of bicarbonate is consumed, it can produce 2.4 g/L algal biomass. If more bicarbonate is consumed, the algae biomass yield can be higher. However, the algae culture is usually limited by light source, and a single culture process may produce limited algae biomass density, and leave a significant concentration of bicarbonate, which can be used for another cycle of algae culture. Thus, repeat culture may be used in this algae culture process. In this situation, the cultured algal biomass is separated and harvested, and the water is discharged into another algae culture system for another round of algae culture.

The invention thus provides a method for CO₂ capture and algae culture, and systems in which the method can be implemented. The method comprises the following steps or processes:

1) a CO₂ capture process that converts CO₂ into bicarbonate; 2) transport of the produced bicarbonate to one or more algae culture system as a water solution or as solid bicarbonate salts; 3) culture of alkaliphilic algae or cyanobacteria in the algae culture system with the transported bicarbonate as the one of carbon sources to produce algal or cyanobacteria bioproducts, and 4) transport of the used (leftover) water from algae culture process for CO₂ capture. In some embodiments, the bicarbonate is present as a salt, e.g. a sodium salt. However, other salts (e.g. potassium, ammonium, etc.) may also be used. Thus, in various embodiments, the bicarbonate solution or salt may be, for example sodium bicarbonate or potassium bicarbonate or ammonium bicarbonate, or even a mixture of these.

The CO₂ capture process is generally a method that produced bicarbonate as one of its product. These methods include, but are not limited to, using a salt of carbonate as an absorbent, or using carbonic anhydrase as a catalyst, or using ammonia and sodium chloride as feedstock to produce bicarbonate, for example, Solvay process and Hou's process (Plasynski et al., 2009).

Sources of CO₂ which is captured include, but are not limited to, thermal power plant (e.g. coal-, natural gas, or oil-fired plants), fermentation processes, anaerobic digestion processes, ammonia plants, air, exhaust, and any other CO₂ sources.

If the bicarbonate is stored as a liquid solution, transportation methods include, but are not limited to, closed pipelines, or open pipelines, tank trucks, tanks transported by rail, or any other transport methods suitable for liquid. In this embodiment, the bicarbonate solution that is transported has a concentration of at least 0.01 mol/L, for example, a range of about 0.01 mol/L to a fully saturated sodium bicarbonate solution. In some embodiments, a preferred concentration is in the range of about 0.3 mol/L up to saturation, i.e. up to a saturated solution of sodium bicarbonate. Those of skill in the art will recognize that saturation is the point at which a solution of a substance can dissolve no more of that substance and additional amounts of it will appear as a precipitate. This point of maximum concentration, the saturation point, may depend on the temperature of the liquid such that if the substance is dissolved to the point of saturation in hot solvent, a change in conditions (e.g. cooling) may result in a supersaturated solution. In some embodiments, the bicarbonate is solid bicarbonate salt. In this embodiment, transportation methods include, but are not limited to, truck, railway, belt, or any other transport methods for solids.

The culture system that utilizes the bicarbonate as a carbon source may be any phototrophic microorganism or group of microorganisms that can utilize bicarbonate as a carbon source. In some embodiments, the phototrophic microorganism are alkaliphilic algae and/or cyanobacteria. Exemplary alkaliphilic algae or cyanobacteria include, but are not limited to, cyanobacteria such as Synechocystis sp., Cyanothece sp., Microcoleus sp., Euhalothece sp., Spirulina sp., eukaryotic microalgae Chlorella and Dunaliella. The microorganisms may be isolated from natural sources, or, alternatively, may be genetically engineered using recombinant techniques, e.g. to increase their tolerance of bicarbonate and/or alkalinity. In some embodiments, the alkaliphilic algae or cyanobacteria include all phototrophic microorganisms that are capable of growing in (i.e. that tolerate) culture medium with a concentration of least about 0.01 mol/L bicarbonate. In some embodiments, the alkaliphilic algae or cyanobacteria include phototrophic microorganisms that grow in culture medium with a concentration range from about 0.01 mol/L bicarbonate up to a saturated solution of bicarbonate. For example, the concentration range may be from at least about 0.3 mol/L bicarbonate up to a saturated solution of bicarbonate.

pH of the culture of alkaliphilic algae or cyanobacteria is generally in a pH range of from about 8.0 to about 12, for example, from about 9.0 to about 11. The alkaliphilic algae or cyanobacteria culture may or may not have a pH control mechanism in the algae culture system, and may or may not have a CO₂ bubbling system.

In some embodiments of the invention, the culture of alkaliphilic algae or cyanobacteria may utilize bicarbonate as the solely carbon source. However, culture systems which include other carbon sources are also encompassed, i.e. bicarbonate may be one of a plurality of carbon sources.

Exemplary culture systems include but are not limited to: open pond systems, closed photo-bioreactor systems, etc. Any suitable culture system may be used in the practice of the invention. In some embodiments, the culture of alkaliphilic algae or cyanobacteria is conducted in batch culture, or in semi-continuous culture, or in continuous culture. Bicarbonate enriched solution (e.g. water) may be used, for example, in one or in multiple steps or batches of the procedure. For example, the culture of alkaliphilic algae or cyanobacteria may use bicarbonate enriched water in more than one batch (i.e. repeat culture) with the same water, e.g. until the pH has increased to a range that algae species cannot survive in.

Used (spent, leftover, residual, etc.) liquid or medium (usually water) from the algae culture system e.g. after the microorganisms have been harvested or otherwise removed from the culture, is also recycled into the system. This spent medium has a pH of between about 8.0 to about 12.0, for example, a pH between about 9.0 to about 11.0, and contains large amounts of carbonate (and also may contain various dissolved salts, minerals, and organic molecular byproducts of microorganism growth, etc.) The carbonate-rich spent medium may be reprocessed to recapture the carbonate, either in solid form, or in a liquid (e.g. water) with a very high carbonate concentration, including and up to saturation, as described above. Transportation of the used water to a suitable processing facility may be carriod our using any suitable approach, for example, a closed pipeline, an open pipeline, or any other transport methods suitable for liquid.

Those of skill in the art will recognize that many products may be produced as the result of the culture of alkaliphilic algae or cyanobacteria produced as described herein. One or more algal products may be produced from a single culture, these products including, but not limited to, algae oil for biofuel, algae oil for nutraceuticals (such as omega-3 fatty acids), pigments (such as carotenoids), alginate, fertilizer, and any other byproducts that can be produced from algae. The invention also encompasses products made by algae or cyanobacteria using the methods and systems of the invention.

The foregoing Examples are provided to illustrate various exemplary embodiments of the invention, but should not be construed so as to limit the invention in any way.

### EXAMPLES

### Example 1

### 1. Strains and Medium

Dunaliella primolecta (UTEX LB 1000) is cultured with artificial seawater medium (UTEX) with reduced concentration of calcium (5% of original concentration) and magnesium (10% of original concentration).

### 2. Well-Plate Culture

The cells were cultured in the 24-well plate, 2 mL for each well. The culture room temperature was controlled at 20° C. Different concentrations of sodium bicarbonate were used as the inorganic carbon source, and no CO2 gas is delivered into the culture. The optical distribution was tested with 750 nm wavelength light for each sample.

Dunaliella primolecta grows to its maximum growth at 3^{rd} day of culture (FIG. 2). The pH was further increased after 3 days culture, when the pH was greater than 10.0, and the final pH in some of the cultures were close to 10.5. Also, its growth in 0.3 M bicarbonate was at the same level as that with lower concentration, but 0.6 M bicarbonate resulted in poor growth. This result indicated that D. primolecta is tolerant to sodium bicarbonate concentration of 0.3M, and it is tolerant to high pH up to 10.5.

### 3. Culture in Photobioreactor

The cells were culture in 250-mL scale photobioreactors, and artificial seawater medium with reduced concentration of calcium (5% of original concentration) and magnesium (10% of original concentration). The culture room temperature was controlled at 20° C. 0.3M sodium bicarbonate was used as the inorganic carbon source in the stirred cultures without bubbling. Two other cultures conducted as comparisons. One group used the same medium, and sparged with 2% (v/v) CO₂ in air. The other group used same medium without bicarbonate, and sparged with 2% (v/v) CO₂ in air.

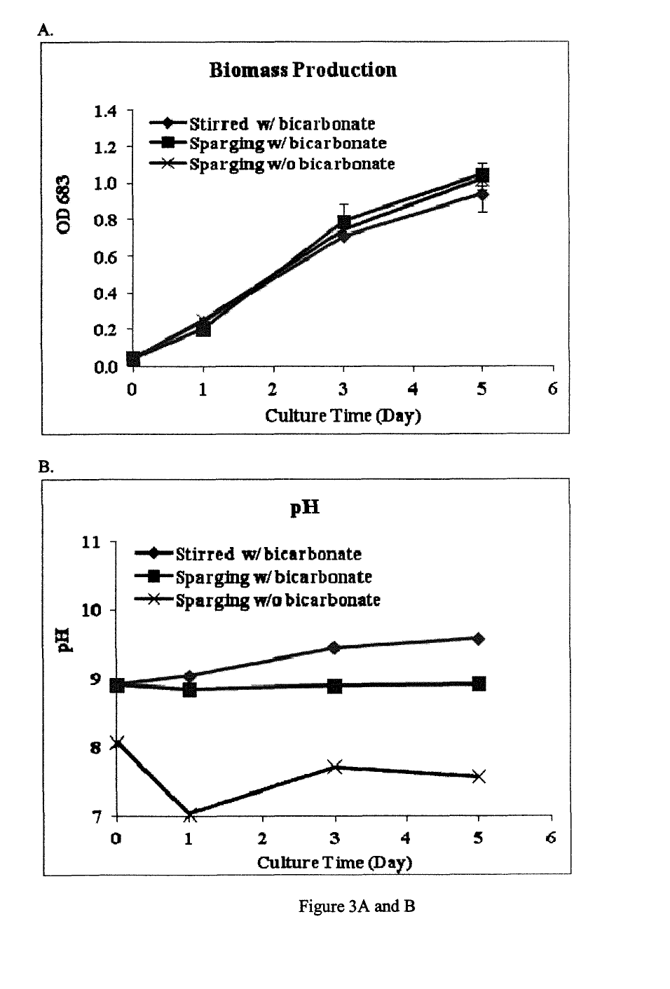

The stirred culture without CO₂ sparging had same productivity as the culture with the two CO₂ sparging controls, with or without bicarbonate as the extra carbon source (FIG. 3). This indicated that sodium bicarbonate can be used as solely carbon source, along with simple stirring. CO₂ sparging cultures had stable pH, but the pH of stirred culture without CO₂ sparging increased gradually. This alkaline water can be used to absorb more CO₂ and supplied to the culture again.

### Example 2

### 1. Strains and Medium

Euhalothece ZM001 is cultured with a 1.0 M sodium bicarbonate concentration, and its compositions are:

| Composition | Concentration | | Reference |
|---|---|---|---|
| NaHCO₃ | 84 | g/L | |
| KNO₃ | 2.5 | g/L | |
| KCl | 2 | g/L | |
| Na2SO4 | 1.4 | g/L | |
| K2HPO4 | 0.38 | g/L | |
| A5 trace element | 1 | mL/L | (Mikhodyuk et al., 2008) |

| | |
|---|---|
| pH | 9.5 |

### 2. Culture in Photo-Bioreactor

The cells were cultured in photobioreactors with agitation, but not aeration. The light path for the photobioreactor was about 0.5 cm, and the photobioreactors were place under the light with intensity of 100 μmol/m²/s. the culture temperature was 35° C.

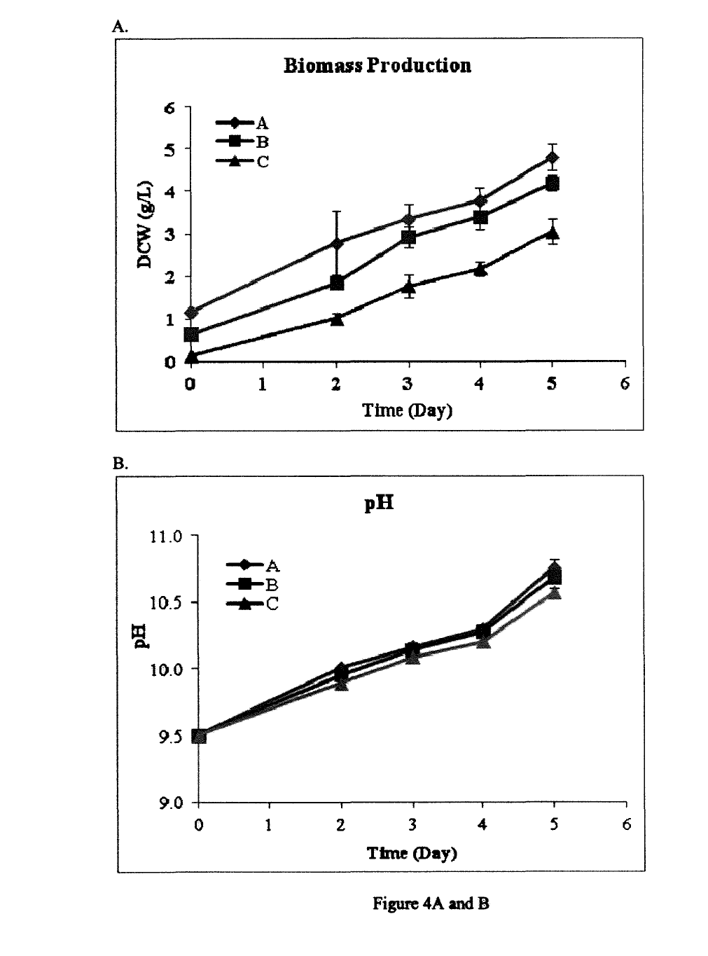

The initial pH was adjusted to 9.5 with sodium hydroxide. With inoculation concentration of 1.2 g/L, the final biomass concentration in this culture was 4.8 g/L, and the daily productivity was 0.72 g/L/day (FIG. 4). The pH in this culture increased to 10.75 after 5 days culture, and this culture medium can be used to absorb more CO₂.

### CONCLUSION

These examples show that sufficient carbon source can be delivered to the algae culture system as bicarbonate, instead of CO₂ gas. The productivity of algae biomass with bicarbonate as inorganic carbon source obtained is at the same level as the culture with CO₂ gas as inorganic carbon source. Culture of Euhalothece sp. used medium contains 1.0 M sodium bicarbonate. This concentration was proved to be effective when carbonate is used as absorbent for CO₂ capture (Plasynski et al., 2009). The productivity of biomass can reach 0.72 g/L/day, and this indicates captured carbon can be utilized efficiently and be converted into algae biomass.

### REFERENCES

Benemann, J., 2009. Growth and Productivity of Algae Biomass Algae Biomass Summit, San Diego.
Chen, S., Chi, Z., O'Fallon, J. V., Zheng, Y., Chakraborty, M., Laskar, D. D., 2010. System integration for producing microalgae as biofuel feedstock. Biofuels, 1, 889-910.
Chisti, Y., 2007. Biodiesel from microalgae. Biotechnology Advances, 25, 294-306.
DOE&EPA, 2000. Carbon Dioxide Emissions from the Generation of Electric Power in the United States.
EPA, 2011. Inventory of U.S. greenhouse gas emissions and sinks: 1990-2009. EPA 430-R-11-005, Washington, D.C.
Fleming, E. D., Prufert-Bebout, L., 2010. Characterization of cyanobacterial communities from high-elevation lakes in the Bolivian Andes. Journal of Geophysical Research-Biogeosciences, 115.
Furukawa, S. K., Bartoo, R. K., 1997 Improved Benfield Process for Ammonia Plants, Des Plaines, Ill.
Gerasimenko, L. M., Mikhodyuk, O. S., 2009. Halophilic Algal-Bacterial and Cyanobacterial Communities and Their Role in Carbonate Precipitation. Paleontological Journal, 43, 940-957.
Iglesias-Rodriguez, M. D., Halloran, P. R., Rickaby, R. E. M., Hall, I. R., Colmenero-Hidalgo, E., Gittins, J. R., Green, D. R. H., Tyrrell, T., Gibbs, S. J., von Dassow, P., Rehm, E., Armbrust, E. V., Boessenkool, K. P., 2008. Phytoplankton calcification in a high-CO2 world. Science, 320, 336-340.
Kadam, K. L., 1997. Plant flue gas as a source of CO2 for microalgae cultivation. Economic impact of different process options. Energy Conversion and Management, 38, S505-S510.
Lal, R., 2004. Soil carbon sequestration impacts on global climate change and food security. Science, 304, 1623-1627.
Lee, S. W., Park, S. B., Jeong, S. K., Lim, K. S., Lee, S. H., Trachtenberg, M. C., 2010. On carbon dioxide storage based on biomineralization strategies. Micron, 41, 273-282.
Mikhodyuk, O. S., Zavarzin, G. A., Ivanovsky, R. N., 2008. Transport systems for carbonate in the extremely natronophilic cyanobacterium Euhalothece sp. Microbiology, 77, 412-418.
NETL, 2010. Cost and Performance Baseline for Fossil Energy Plants Volume 1: Bituminous Coal and Natural Gas to Electricity, Revision 2, November 2010. Department of Energy, pp. DOE/2010/1397.
Oberholster, P. J., Botha, A. M., Ashton, P. J., 2009. Appearance of new taxa: invertebrates, phytoplankton and bacteria in an alkaline, saline, meteorite crater lake, South Africa. Fundamental and Applied Limnology, 174, 271-282.
Packer, M., 2009. Algal capture of carbon dioxide; biomass generation as a tool for greenhouse gas mitigation with reference to New Zealand energy strategy and policy. Energy Policy, 37, 3428-3437.
Pienkos, Darzins, 2009. The promise and challenges of microalgal-derived biofuels. Biofuels, Bioproducts and Biorefining, 3, 431-440.
Pikuta, E. V., Hoover, R. B., Tang, J., 2007. Microbial extremophiles at the limits of life. Critical Reviews in Microbiology, 33, 183-209.
Plasynski, S. I., Litynski, J. T., McIlvried, H. G., Srivastava, R. D., 2009. Progress and New Developments in Carbon Capture and Storage. Critical Reviews in Plant Sciences, 28, 123-138.
Price, G. D., Badger, M. R., Woodger, F. J., Long, B. M., 2008. Advances in understanding the cyanobacterial CO2-concentrating-mechanism (CCM): functional components, Ci transporters, diversity, genetic regulation and prospects for engineering into plants. Journal of Experimental Botany, 59, 1441-1461.
Ramanan, R., Kannan, K., Deshkar, A., Yadav, R., Chakrabarti, T., 2010 Enhanced algal CO2 sequestration through calcite deposition by Chlorella sp and Spirulina platensis in a mini-raceway pond. Bioresource Technology, 101, 2616-2622.
Sheehan, J., Dunahay, T., Benemann, J., Roessler, P., 1998. A Look Back at the U.S. Department of Energy's Aquatic Species Program: Biodiesel from Algae. National Renewable Energy Laboratory.
Sminchak, J., Gupta, N., 2001. Issues related to seismic activity induced by the injection of CO2 in deep saline aquifers. Accessed on May 19, 2011 ed. in: A.o. May 19, 2011 (Ed.). Accessed May 19, 2011 on, pp. Accessed on May 19, 2011.
Zavarzin, G. A., Zhilina, T. N., Kevbrin, V. V., 1999. The alkaliphilic microbial community and its functional diversity. Microbiology, 68, 503-521.
Zhou, Y., Richard, S. J., 2005. Evaluating the costs of desalination and water transport. Water Resources Research, 41 WO3003.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. An integrated method culturing algae or cyanobacteria, comprising the steps of
i) capturing CO₂ from a source of CO₂;
ii) converting captured CO₂ into bicarbonate;
iii) culturing alkaliphilic algae or alkaliphilic cyanobacteria using said bicarbonate as a carbon source to produce algal bioproducts;
iv) using spent medium from said step of culturing as said source of CO₂ in said step of capturing; and
v) repeating steps i) to iv).

2. The method of claim 1, wherein said bicarbonate is in a form selected from the group consisting of solid bicarbonate and a liquid bicarbonate solution.

3. The method of claim 1, wherein said alkaliphilic cyanobacteria are selected from the group consisting of Synechocystis sp., Cyanothece sp., Microcoleus sp., Euhalothece sp. and Spirulina sp.

4. The method of claim 1, wherein said alkaliphilic algae are eukaryotic microalgae selected from the group consisting of Chlorella and Dunaliella.

5. The method of claim 1, wherein culture medium used in said step of culturing has a concentration in a range of from 0.01 mol/L bicarbonate to saturation.

6. The method of claim 5, wherein culture medium used in said step of culturing has a concentration in a range of from 0.03 mol/L bicarbonate to saturation.

7. The method of claim 1, wherein culture medium used in said step of culturing is carried out at a pH of from 8.0 to 12.

8. The method of claim 7, wherein culture medium used in said step of culturing is carried out at a pH of from 9.0 to 11.

9. The method of claim 1, wherein said step of capturing is carried out using a method selected from the group consisting of: using salt of carbonate as absorbent, using carbonic anhydrase as catalyst, and using ammonia and sodium chloride as feedstock to produce bicarbonate.

10. The method of claim 1, wherein said CO₂ source is selected from the group consisting of: thermal power plant emissions, a fermentation process, an anaerobic digestion process, an ammonia plant, and air.

11. The method of claim 2, wherein said liquid bicarbonate solution has a concentration in the range of 0.01 mol/L to saturation.

12. The method of claim 11, wherein said liquid bicarbonate solution has a concentration in the range of 0.3 mol/L to saturation.

13. The method of claim 1, wherein said alkaliphilic algae or said alkaliphilic cyanobacteria utilizes bicarbonate as a sole carbon source.

14. The method of claim 1, wherein said alkaliphilic algae or said alkaliphilic cyanobacteria utilizes bicarbonate as one of more than one carbon sources.

15. The method of claim 1, wherein said step of culturing is performed in an algae culture system selected from the group consisting of an open pond system and a closed photo-bioreactor system.

16. The method of claim 1, wherein said step of culturing is conducted in batch culture, semi-continuous culture, or continuous culture.

17. The method of claim 1, wherein said bicarbonate is a salt selected from the group consisting of sodium, potassium and ammonium.

18. The method of claim 1, further comprising the step of obtaining from said alkaliphilic algae or said alkaliphilic cyanobacteria a product selected from the group consisting of algae oil for biofuel, algae oil for nutraceuticals, omega-3 fatty acids, pigments, carotenoids, alginate, and fertilizer.

19. The method of claim 1, wherein steps of capturing CO₂ from a source of CO₂ and converting CO₂ into bicarbonate are carried out as a single step.

20. The method of claim 1, further comprising a step vi) of recovering said bioproducts.

21. An integrated system for culturing algae or cyanobacteria, comprising
an apparatus for capturing CO₂ from a source of CO₂;
an apparatus for converting CO₂ to bicarbonate;
a culture system for culturing alkaliphilic algae or alkaliphilic cyanobacteria using said bicarbonate; and
transport means for
i) transporting captured CO₂ from said source of CO₂ to said apparatus for converting CO₂ to bicarbonate; and
ii) transporting bicarbonate from said apparatus for converting CO₂ to bicarbonate to said culture system.

22. The integrated system of claim 21, wherein said culture system optionally comprises a pH control system and/or a CO₂ bubbling system.

23. The integrated system of claim 21, wherein said bicarbonate is in a liquid solution and said transport means is selected from the group consisting of a closed pipeline, an open pipeline, and a tank.

24. The integrated system of claim 21, wherein said bicarbonate is a solid and said transport means is selected from the group consisting of a truck and a railroad car.

25. The integrated system of claim 21, wherein said apparatus for capturing CO₂ from a source of CO₂ and said apparatus for converting CO₂ to bicarbonate are a single apparatus.
